(19) United States
(12) Patent Application Publication
(10) Pub. No.: US 2018/0256011 A1
(43) Pub. Date: Sep. 13, 2018

(54) ENDOSCOPE ADAPTOR, ENDOSCOPE, AND ENDOSCOPE SYSTEM

(71) Applicant: OLYMPUS CORPORATION, Tokyo (JP)

(72) Inventors: Yoshiro OKAZAKI, Tokyo (JP); Naoya SUGIMOTO, Tokyo (JP)

(73) Assignee: OLYMPUS CORPORATION, Tokyo (JP)

(21) Appl. No.: 15/973,631

(22) Filed: May 8, 2018

Related U.S. Application Data
(63) Continuation of application No. PCT/JP2015/083439, filed on Nov. 27, 2015.

(51) Int. Cl. A61B 1/00; A61B 17/02; A61B 1/313; A61B 1/32; A61B 90/00; A61B 1/018

(52) U.S. Cl. CPC A61B 1/00087; A61B 17/0218; A61B 1/313; A61B 2017/0225; A61B 90/02; A61B 1/0014; A61B 1/018; A61B 1/32

(57) ABSTRACT

To more reliably secure the space in the pericardial cavity to comply with the change in viewing area range of an endoscope, an endoscope adaptor is attached to an endoscope having a bending portion near a distal end, the endoscope adaptor including a projecting portion that projects from a position on a proximal end side of the bending portion so as to project in one part in a circumferential direction of the endoscope and in a direction along a longitudinal axis of the endoscope with the bending portion in an un-bent state; and a guide through hole, which is formed in the projecting portion and penetrates through the projecting portion in the projecting direction and through which a long thin guide member for guiding the endoscope to a desired observation site can pass in the projecting direction.

This is a continuation of International Application PCT/JP2015/083439, with an international filing date of Nov. 27, 2015, which is hereby incorporated by reference herein in its entirety.

### TECHNICAL FIELD

The present invention relates to an endoscope adaptor, an endoscope and an endoscope system.

### Background Art

An endoscope system that has a bending portion at its distal end and is used to observe an affected site by percutaneously inserting an endoscope and a treatment tool from under the ensiform cartilage into the pericardial cavity without performing open-heart surgery is known (for example, refer to PTL 1).

With this endoscope system, a space-securing device is placed in the gap between the heart and the pericardium so as to create the space therebetween so that the operability can be improved by securing the space necessary for operating the endoscope and the treatment tool.

### CITATION LIST

### Patent Literature

### {PTL 1} Japanese Unexamined Patent Application Publication No. 2011-67600

### SUMMARY OF INVENTION

According to an aspect of the present invention, there is provided an endoscope adaptor attached to an endoscope having a bending portion near a distal end, the endoscope adaptor including a projecting portion that projects from a position on a proximal end side of the bending portion so as to project in one part in a circumferential direction of the endoscope and in a direction along a longitudinal axis of the endoscope with the bending portion in an un-bent state; and a guide through hole, which is formed in the projecting portion and penetrates through the projecting portion along the projecting direction and through which a long thin guide member for guiding the endoscope to a desired observation site can pass in the projecting direction.

According to another aspect of the present invention, there is provided an endoscope including an insertion portion having a bending portion near a distal end; a projecting portion that projects from a position on a proximal end side of the bending portion so as to project in one part in a circumferential direction of the insertion portion and in a direction along a longitudinal axis of the insertion portion with the bending portion in an un-bent state; and a guide through hole, which is formed in the projecting portion and penetrates through the projecting portion in the projecting direction and through which a long thin guide member can pass in the projecting direction, wherein the projecting portion is integrally fixed to the insertion portion.

According to yet another aspect of the present invention, there is provided an endoscope system including the endoscope adaptor described above and the endoscope.

According to yet another aspect of the present invention, there is provided an endoscope system including the endoscope adaptor described above; and the endoscope, in which the endoscope adaptor has a tubular sheath through which the endoscope is passed, and the projecting portion is provided at a distal end portion of the sheath.

### BRIEF DESCRIPTION OF DRAWINGS

FIG. 1 is a side view of an endoscope system according to one embodiment of the present invention.

FIG. 2 is a perspective view of a distal end portion of the endoscope system illustrated in FIG. 1.

FIG. 3 is a longitudinal sectional view of a viewing area-securing adaptor according to one embodiment of the present invention, provided to the endoscope system.

FIG. 4 is a diagram illustrating the state of observing the pericardial cavity by the endoscope system.

FIG. 5 is a side view of a modification of the endoscope system illustrated in FIG. 1.

FIG. 6 is a side view of another modification of the endoscope system illustrated in FIG. 1.

FIG. 7 is a side view of a modification of the endoscope system illustrated in FIG. 6.

FIG. 8 is a perspective view of a distal end portion illustrating a modification of the endoscope system illustrated in FIG. 1.

FIG. 9 is a plan view illustrating a portion of a modification of a guide wire provided to the endoscope system illustrated in FIG. 1.

FIG. 10 is a partial breakaway view illustrating a part of another modification of the guide wire provided to the endoscope system illustrated in FIG. 1.

FIG. 11 is a perspective view of a distal end portion illustrating another modification of the endoscope system illustrated in FIG. 1.

FIG. 12 is a longitudinal sectional view illustrating a state in which a space has been secured inside the pericardial cavity by the guide wire provided to the endoscope system illustrated in FIG. 11.

FIG. 13 is a longitudinal sectional view illustrating a state in which the guide wire in the state illustrated in FIG. 12 is rotated to move the space.

### DESCRIPTION OF EMBODIMENTS

A viewing area-securing adaptor 2 and an endoscope system 100 according to one embodiment of the present invention will now be described with reference to the drawings.

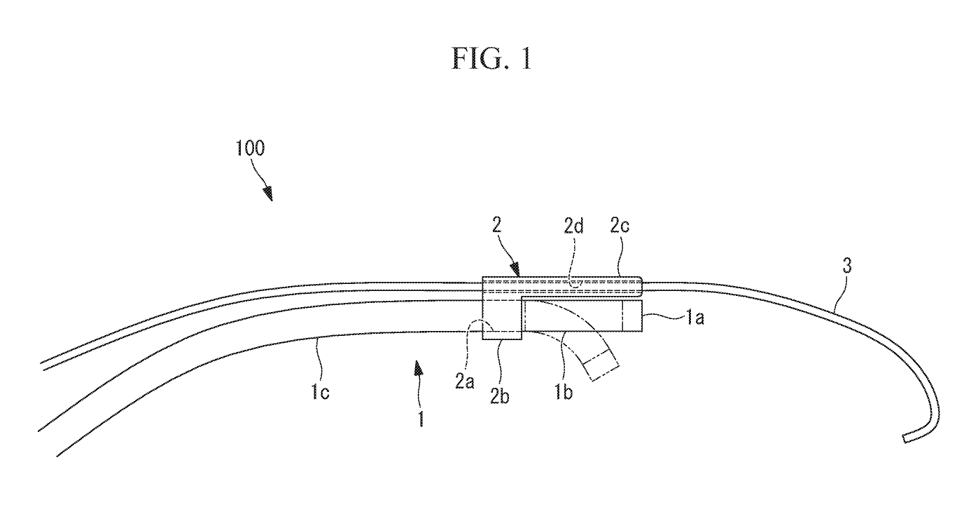

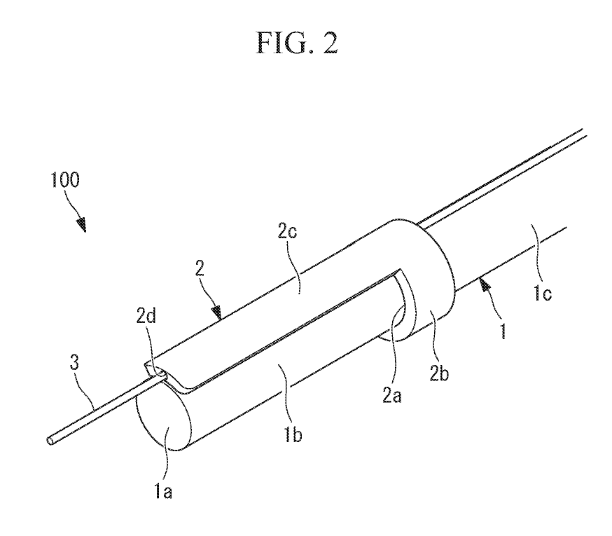

As illustrated in FIGS. 1 and 2, the endoscope system 100 according to this embodiment includes an endoscope 1 percutaneously inserted into the pericardial cavity B, a viewing area-securing adaptor (endoscope adaptor) 2 fixed to the distal end of the endoscope 1, and a guide wire (guide member) 3 that guides the endoscope 1 into the pericardial cavity B.

The endoscope 1 is a direct-view-type flexible endoscope that observes a viewing area in front of a distal end surface 1a. The endoscope 1 is equipped with a long thin insertion portion 1c having a bending portion 1b at its distal end portion, and an operation unit (not illustrated) connected to a proximal end of the insertion portion 1c. The image acquired by the endoscope 1 is displayed in a display unit not illustrated in the drawings.

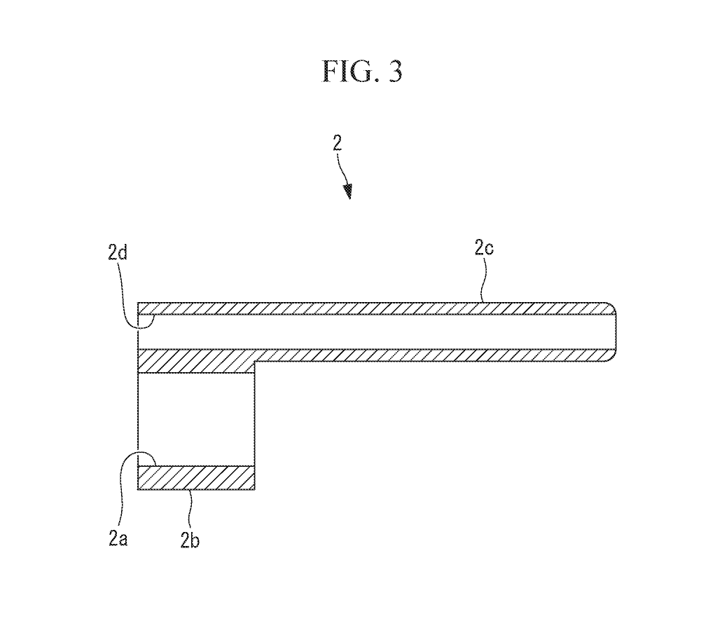

As illustrated in FIGS. 1 to 3, the viewing area-securing adaptor 2 according to an embodiment of the present invention includes a ring portion 2b having an endoscope through hole 2a, through which the insertion portion 1c of the endoscope 1 is passed, and a projecting portion 2c that projects from one site in the circumferential direction of the ring portion 2b so as to project parallel to the longitudinal axis direction of the endoscope through hole 2a. The projecting portion 2c has a guide through hole 2d, which penetrates through the projecting portion 2c in a direction parallel to the longitudinal axis of the endoscope through hole 2a and through which the guide wire 3 is passed.

The viewing area-securing adaptor 2 is fixed on the proximal end side of the bending portion 1b of the endoscope 1 while having the endoscope 1 passing through the endoscope through hole 2a. In this manner, the projecting portion 2c extends in a straight line from the proximal end side of the bending portion 1b of the endoscope 1 toward the distal end side, and lies on one side 1 in the circumferential direction with respect to the center axis of the endoscope. In this state, the distal end surface 1a of the endoscope 1 can be moved to the side where the projecting portion 2c does not exist by operating the bending portion 1b.

As illustrated in FIG. 2, the projecting portion 2c is formed to have an arced plate shape formed by extending, in the longitudinal axis direction, one portion of the ring portion 2b in the circumferential direction, and the corners are chamfered so that the projecting portion 2c has a rounded form.

The operation of the endoscope system 100 of this embodiment configured as such will now be described.

In order to observe the heart A by using the endoscope system 100 of this embodiment, first, the guide wire 3 is percutaneously inserted from under the ensiform cartilage into the pericardial cavity B. Insertion of the guide wire 3 is achieved by using a puncture needle (not illustrated), etc.

While the distal end of the guide wire 3 is placed in the pericardial cavity B, the endoscope 1 is inserted.

Insertion of the endoscope 1 is conducted by using a sheath (not illustrated) and a dilator (not illustrated). The dilator is a long thin member insertable into the insertion hole of the sheath in the longitudinal direction, and has a distal end portion having a substantially conical shape with the diameter gradually decreasing toward the distal end. The dilator has a hole for the guide wire 3, the hole penetrating in the longitudinal direction.

The dilator is inserted into the sheath so that the distal end portion of the dilator projects from the distal end of the sheath so as to install the dilator onto the sheath, and, while having the guide wire 3 pass through the interior of the dilator, the dilator and the sheath are advanced together along the guide wire 3.

Since the distal end portion of the dilator has a substantially conical shape that gradually becomes thicker from the distal end toward the proximal end, the dilator and the sheath can be easily inserted into the pericardial cavity B while a fine hole in the pericardium C through which the guide wire 3 passes is gradually expanded by the distal end portion of the dilator. After the sheath is inserted to reach the observation site in the pericardial cavity B, the dilator is removed while leaving the sheath in the pericardial cavity B.

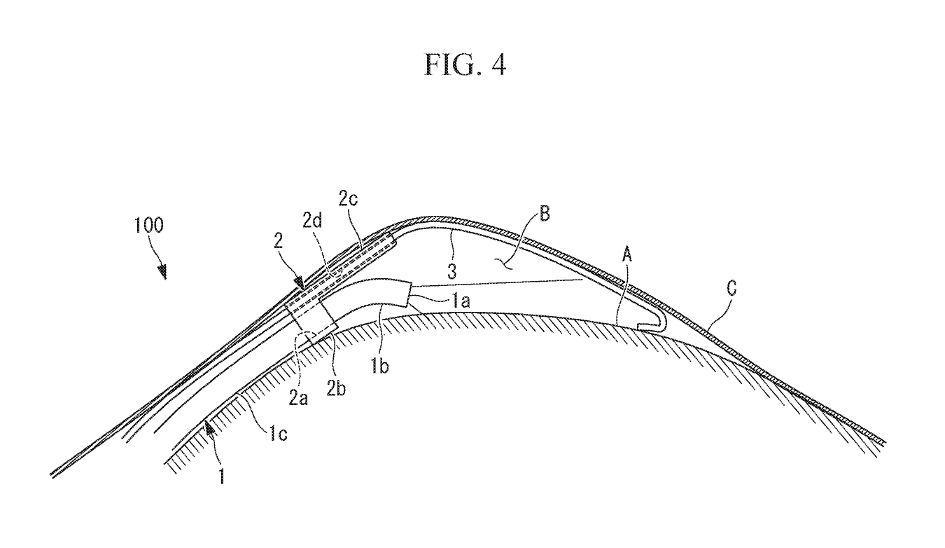

Next, the guide wire 3 is passed through the guide through hole 2d, and the endoscope 1 equipped with the viewing area-securing adaptor 2 is inserted along the guide wire 3 through the interior of the sheath into the pericardial cavity B. The rotation angle about the longitudinal axis of the endoscope 1 is adjusted so that the projecting portion 2c of the viewing area-securing adaptor 2 arrives at the pericardium C side. Next, the operation unit of the endoscope 1 is operated so that the bending portion 1b bends toward the heart A side. As a result, as illustrated in FIG. 4, the surface of the heart A can be observed by the endoscope 1 while the projecting portion 2c presses the pericardium C.

In other words, according to the endoscope system 100 of this embodiment, because the projecting portion 2c of the viewing area-securing adaptor 2 supports the pericardium C to prevent the pericardium C from approaching the surface of the heart A, the projecting portion 2c serves as eaves to secure a space between the projecting portion 2c and the surface of the heart A. In this state, when the bending portion 1b of the endoscope 1 disposed on the heart A side is bent toward the heart A side, the distal end surface 1a of the endoscope 1 can be arranged to oppose the surface of the heart A in the space secured by the projecting portion 2c, and thus, there is an advantage in that the surface of the heart A can be panoramically observed by the endoscope 1.

Furthermore, when the endoscope 1 is passed through the endoscope through hole 2a in the viewing area-securing adaptor 2, the endoscope 1 and the projecting portion 2c can be moved simultaneously due to the projecting portion 2c fixed to the endoscope 1, which advantageously makes scanning easier. Since the surface of the heart A is observed by pressing the pericardium C with the projecting portion 2c to secure the space between the pericardium C and the surface of the heart A, the projecting portion 2c comes on the rear surface side in the viewing area of the endoscope 1. Thus, the guide wire 3 that passes through the guide through hole 2d in the projecting portion 2c and extends forward does not obstruct the viewing area of the endoscope 1.

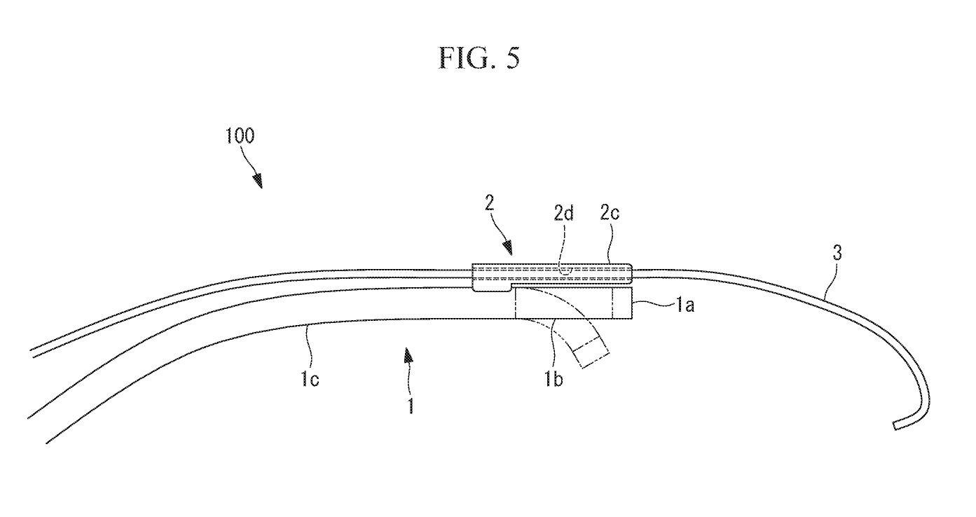

In this embodiment, the viewing area-securing adaptor 2 is to be detachably attached to a portion near the distal end of the insertion portion 1c of the endoscope 1; alternatively, as illustrated in FIG. 5, the projecting portion 2c may be formed to be integrated with the insertion portion 1c of the endoscope 1 so that the projecting portion 2c extends forward from the proximal end side of the bending portion 1b of the endoscope 1. In this manner, misalignment between the endoscope 1 and the viewing area-securing adaptor 2 can be more reliably prevented.

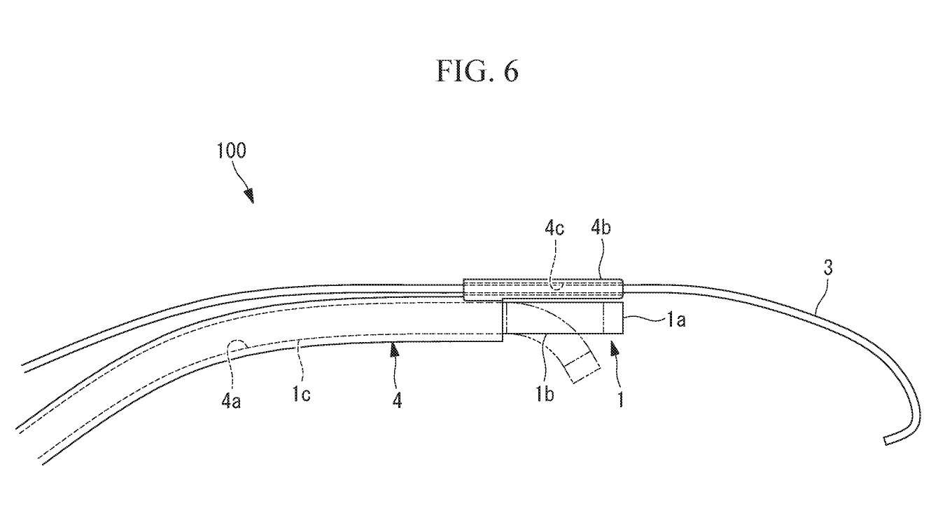

In this embodiment, as illustrated in FIG. 6, the endoscope system 100 may include a sheath 4 having an inner hole 4a through which the endoscope 1 passes, and a projecting portion 4b may be provided at one site of the distal end surface of the sheath 4 in the circumferential direction so as to extend forward. In this manner, as illustrated in FIG. 6, the projecting portion 4b provided on the sheath 4 can function as described above by causing the endoscope 1 to project from the distal end of the sheath 4 so as to expose the bending portion 1b.

As illustrated in FIG. 6, the guide through hole 4c that penetrates through the projecting portion 4b in the longitudinal direction may be provided in the distal end of the sheath 4 only, or may be provided across the entire length of the sheath 4. When the guide through hole 4c is provided in the distal end of the sheath 4 only, an advantage is offered in that a short guide wire 3 can be used.

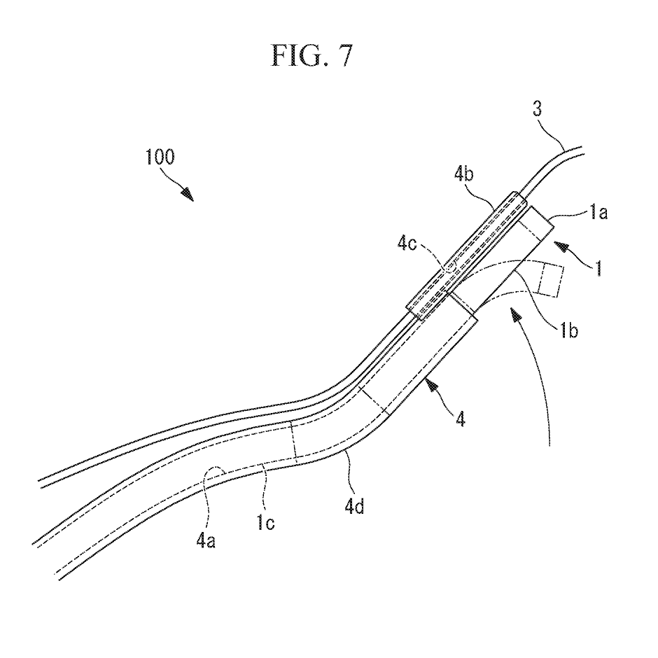

Moreover, as illustrated in FIG. 7, a bending portion 4d may be provided to the sheath 4. In this manner, the pericardium C can be lifted by pressing the projecting portion 4b toward the pericardium C side by causing the bending portion 4d of the sheath 4 to bend, and, thus, a larger viewing space can be secured.

In this embodiment, a common endoscope 1 can be used as the endoscope 1. In such a case, the bending portion 1b can be bent in any desired direction, but the bending in the direction of the projecting portion 2c of the bending portion 1b is limited by the projecting portion 2c. Thus, the bending direction may be restricted such that only bending toward the opposite side of the projecting portion 2c is possible.

The guide wire 3 used in this embodiment preferably itself has stiffness sufficient to press and spread the pericardium C. In this manner, not only the projecting portion 2c but also the guide wire 3 can be used to press and expand the space between the pericardium C and the surface of the heart A, and, thus, a larger viewing space can be secured. The surface of the heart A can be panoramically observed from a position remote from the surface of the heart A by advancing the endoscope 1 along the guide wire 3 that is pressing and spreading the pericardium C.

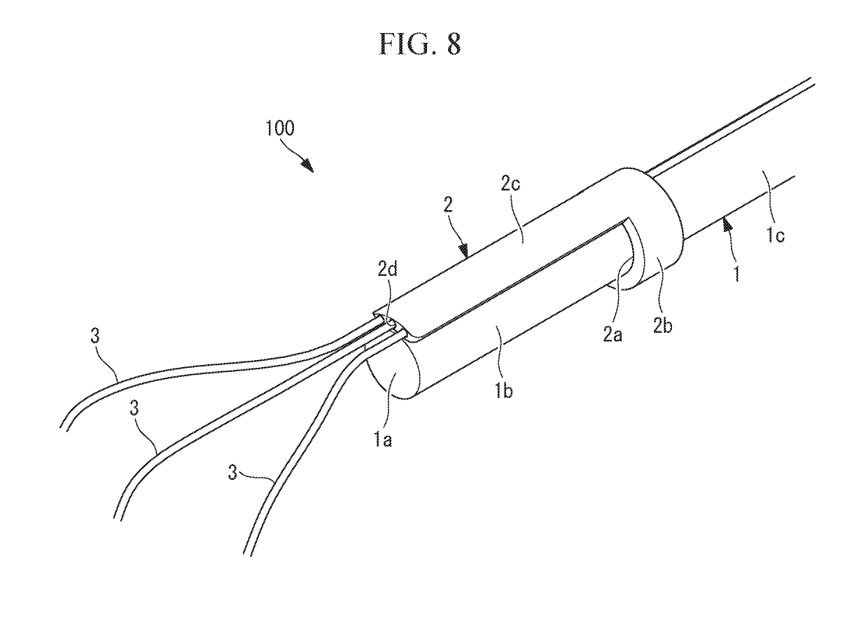

In this embodiment, the case in which only one guide wire 3 is provided has been described, but the number of guide wires is not limited to this. Two or more guide wires 3 may be provided. As illustrated in FIG. 8, this is advantageous since guide wires 3 projecting from the distal end of the projecting portion 2c are preliminarily given a tendency to extend and spread in different directions so as to press and spread the pericardium C over a wide range, and, thus, a larger viewing space can be secured.

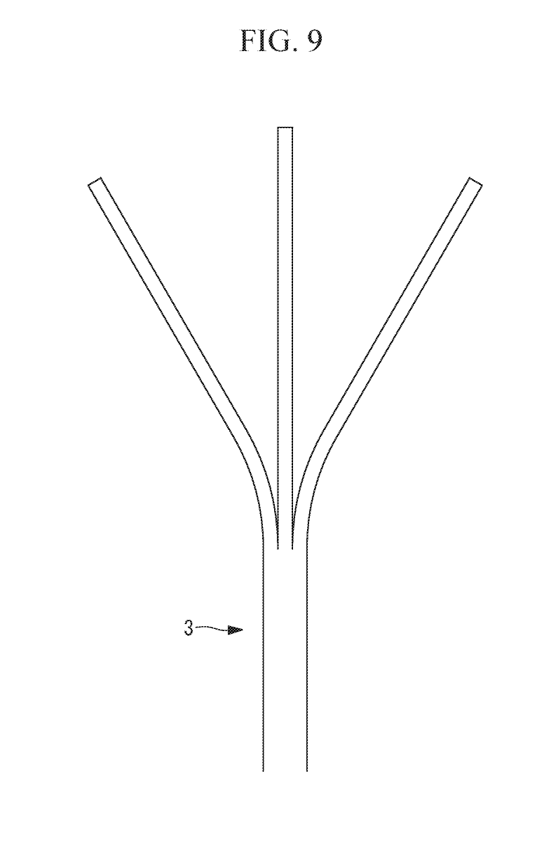

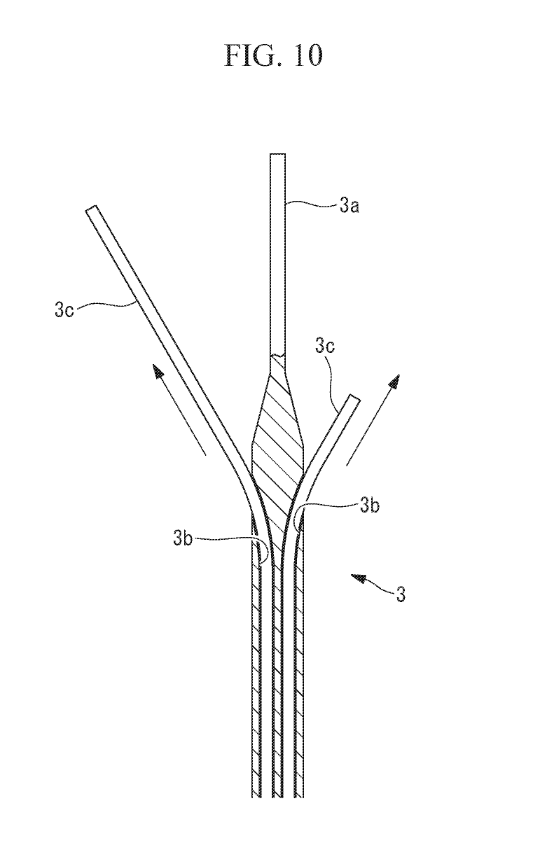

Examples of the guide wire 3 that can be employed include a guide wire that has a distal end branching into multiple wires (main guide member and auxiliary guide members) as illustrated in FIG. 9, and a guide wire that has a hollow main guide wire (main guide member) 3a having inner holes 3b that have openings in the side surface of the main guide wire 3a, and auxiliary guide wires (auxiliary guide members) 3c that pass through the inner holes 3b and that can go out and come in through the openings, as illustrated in FIG. 10. According to these, when the guide wire 3 is being guided, the ease of insertion is improved because they are a single guide wire 3; however, once inside the pericardial cavity B, multiple wires press and spread the pericardium C over a wide range, and a large viewing space can be secured.

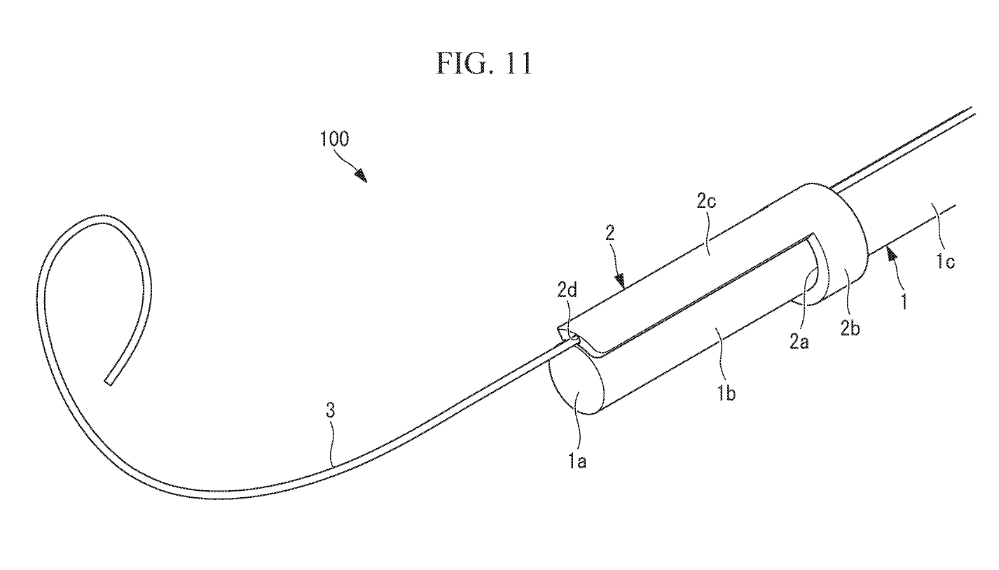

Moreover, as illustrated in FIG. 11, the distal end portion of the single guide wire 3 may be preliminarily given a tendency to bend three-dimensionally. The shape of the portion that bends three-dimensionally may be any shape. For example, the shape may be a shape, such as a coil shape, that prevents the portion from collapsing in any direction due to the pressing force from the pericardium C when the distal end portion is sandwiched between the pericardium C and the heart A. The portion that bends three-dimensionally is unevenly distributed on one side with respect to the axial line of the guide through hole 2d of the projecting portion 2c.

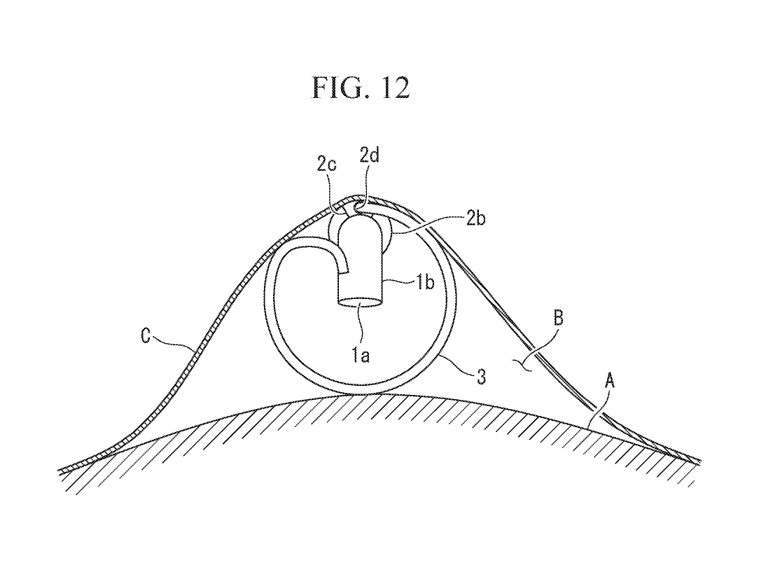

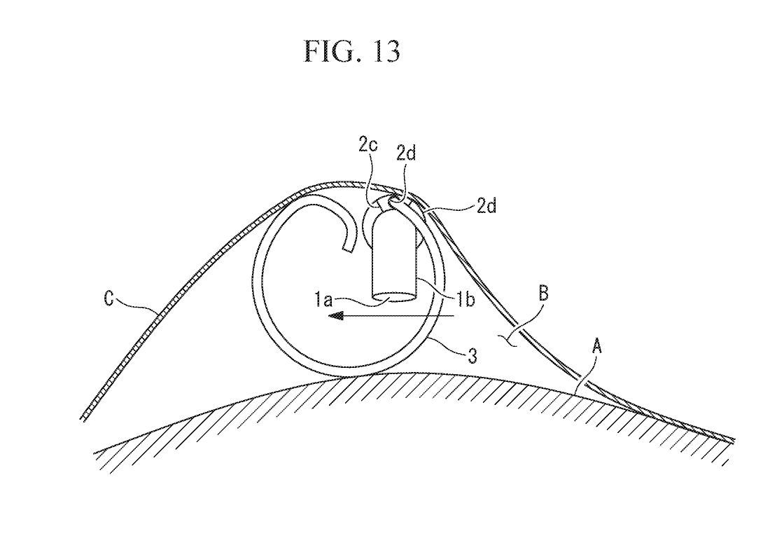

According to this structure, when the guide wire 3 in the state illustrated in FIG. 12 is rotated about the longitudinal axis of the guide wire 3, the portion that bends three-dimensionally placed in the pericardial cavity B can be moved in a direction orthogonal to the axial line of the guide through hole 2d, as illustrated in FIG. 13. As a result, there is an advantage in that the space between the pericardium C and the heart A secured by the portion of the guide wire 3 that bends three-dimensionally as appropriate can be moved in a direction (direction indicated by the arrow) intersecting the longitudinal axis of the endoscope 1.

From the above-described embodiments and modifications thereof, the following aspects of the invention are derived.

According to an aspect of the present invention, there is provided an endoscope adaptor attached to an endoscope having a bending portion near a distal end, the endoscope adaptor including a projecting portion that projects from a position on a proximal end side of the bending portion so as to project in one part in a circumferential direction of the endoscope and in a direction along a longitudinal axis of the endoscope with the bending portion in an un-bent state; and a guide through hole, which is formed in the projecting portion and penetrates through the projecting portion along the projecting direction and through which a long thin guide member for guiding the endoscope to a desired observation site can pass in the projecting direction.

According to this aspect, the long thin guide member is percutaneously inserted from the outside of the body into the pericardial cavity, and then the projecting portion with the guide member passed through the guide through hole is moved along the guide member so as to insert the projecting portion and the endoscope into the pericardial cavity. When the projecting portion is placed at a position such that the projecting portion makes close contact with the pericardium, the projecting portion serves as eaves to secure a space between the projecting portion and the surface of the heart. In this state, when the bending portion of the endoscope disposed on the heart side is bent toward the heart side, the distal end of the endoscope can be arranged to oppose the surface of the heart in the space secured by the projecting portion, and thus, the surface of the heart can be panoramically observed by the endoscope.

In this case, since the projecting portion projects from the proximal end side of the bending portion of the endoscope, moving the endoscope inside the pericardial cavity also causes the projecting portion to move together with the endoscope, and thus, the viewing area space for observation by the endoscope can be secured.

In the above-described aspect, the projecting portion may have an endoscope through hole, which is provided to be substantially parallel to the guide through hole and through which the endoscope is passed, and the endoscope adaptor may be detachably attached to the endoscope.

In this manner, the projecting portion can be integrally attached to the endoscope by passing the endoscope through the endoscope through hole. As a result, when the guide member is passed through the guide through hole and the projecting portion is introduced into the pericardial cavity along the guide member, the distal end portion of the endoscope can be easily placed within the viewing area space secured by the projecting portion.

In the above-described aspect, the bending portion may bend in one direction in the circumferential direction of the endoscope, and the projecting portion may be disposed at a position opposite to the bending direction of the bending portion with respect to the endoscope.

In this manner, the pericardium that lies on the opposite side in the viewing area direction of the endoscope by bending the bending portion is pressed by the projecting portion and retained so as not to approach the endoscope; thus, the viewing area space can be reliably secured.

According to another aspect of the present invention, there is provided an endoscope including an insertion portion having a bending portion near a distal end; a projecting portion that projects from a position on a proximal end side of the bending portion so as to project in one part in a circumferential direction of the insertion portion and in a direction along a longitudinal axis of the insertion portion with the bending portion in an un-bent state; and a guide through hole, which is formed in the projecting portion and penetrates through the projecting portion in the projecting direction and through which a long thin guide member can pass in the projecting direction, wherein the projecting portion is integrally fixed to the insertion portion.

In this manner, when the guide member is passed through the guide through hole and the projecting portion integral with the insertion portion is introduced into the pericardial cavity along the guide member, the distal end portion of the endoscope can be easily placed within the viewing area space secured by the projecting portion.

According to yet another aspect of the present invention, there is provided an endoscope system including the endoscope adaptor described above and the endoscope.

In this manner, within the space secured by the endoscope adaptor, the distal end of the endoscope can be arranged to oppose the surface of the heart, and the surface of the heart can be panoramically observed by the endoscope.

According to yet another aspect of the present invention, there is provided an endoscope system including the endoscope adaptor described above; and the endoscope, in which the endoscope adaptor has a tubular sheath through which the endoscope is passed, and the projecting portion is provided at a distal end portion of the sheath.

In this manner, the bending portion of the endoscope can be placed in front of the distal end portion of the sheath and the projecting portion can be arranged to extend forward from the proximal end side of the bending portion by having the endoscope to pass through the tubular sheath and having the distal end of the endoscope project from the distal end surface. As a result, the distal end of the sheath can be introduced into the pericardial cavity along the guide member first introduced into the pericardial cavity, and then the endoscope can be introduced into the pericardial cavity along the sheath. The distal end portion of the endoscope can be placed within the viewing area space secured by the projecting portion provided on the distal end portion of the sheath, and the surface of the heart can be easily observed.

In the above-described aspect, the sheath may have a sheath bending portion that causes the sheath to bend, the sheath bending portion being disposed near a distal end of the sheath.

In this manner, when the projecting portion of the sheath is brought into close contact with the pericardium and the sheath bending portion is operated, the projecting portion can be moved in a direction away from the surface of the heart, and thus, the space can be more reliably secured between the projecting portion and the surface of the heart.

In the above-described aspect, the guide member may have a main guide member that extends in a direction in which the guide through hole extends, and one or more long thin auxiliary guide members that extend in directions different from the main guide member.

In this manner, since the main guide member and one or more auxiliary guide members extend in different directions within the pericardial cavity, the pericardium can be pressed by these guide members over a wide range, and a larger viewing area space can be secured.

In the above-described aspect, the guide member may be a guide wire preliminarily given tendency to bend three-dimensionally in a portion projecting from the guide through hole.

In this manner, the guide wire disposed forward from the projecting portion is sandwiched between the pericardium and the surface of the heart, and thus, a large viewing area space of the endoscope is secured.

In the above-described aspect, the portion of the guide wire three-dimensionally bent may be disposed while being decentered with respect to the guide through hole in a direction intersecting the longitudinal axis of the guide through hole.

In this manner, when the guide wire is twisted about the longitudinal axis of the guide wire outside the body, the portion that bends three-dimensionally and disposed in the pericardial cavity is rotated about the longitudinal axis of the guide through hole. Since the three-dimensionally bent portion is decentered in one direction intersecting the longitudinal axis, position within the pericardial cavity can be moved by the rotation. As a result, the viewing area space secured in the pericardial cavity can be easily shifted to expand the observation range.

According to the present invention, a space within the pericardial cavity can be more reliably secured to comply with changes in the viewing area range of the endoscope.

### REFERENCE SIGNS LIST

1 endoscope
1b bending portion
1c insertion portion
2 viewing area-securing adaptor (endoscope adaptor)
2a endoscope through hole
2c projecting portion
2d guide through hole
3 guide wire (guide member)
3a main guide wire (main guide member)
3c auxiliary guide wire (auxiliary guide member)
4 sheath
4d sheath bending portion
100 endoscope system
A heart
B pericardial cavity
C pericardium

1} An endoscope system comprising:
an endoscope that observes a viewing area in front of the distal end surface, the endoscope being provided with a insertion portion having a longitudinal axis and a bending portion that is disposed near the distal end and that bends respect to the longitudinal axis with the insertion portion in an un-bent state;
a projecting portion that projects from a position on a proximal end side of the bending portion of the insertion portion to a projecting direction along the longitudinal axis and that is disposed at one part along a circumferential direction of the insertion portion in a side different from a direction in which the bending portion is bended;
a guide through hole, which is formed in the projecting portion and penetrates through the projecting portion in the projecting direction and through which a long thin guide member for guiding the insertion portion to a desired observation site can pass in the projecting direction.

2} The endoscope system according to claim 1, wherein the bending portion bends in one direction in the circumferential direction of the insertion portion, and
the projecting portion is disposed at a position opposite to the bending direction of the bending portion with respect to the endoscope.

3} The endoscope system according to claim 1, wherein the projecting portion is provided to be substantially parallel to the guide through hole.

4} The endoscope system according to claim 1, wherein the guide member has a main guide member that extends in a direction in which the guide through hole extends, and one or more long thin auxiliary guide members that extend in directions different from the main guide member.

5} The endoscope system according to claims 1, wherein the guide member is a guide wire preliminarily given tendency to bend three-dimensionally in a portion projecting from the guide through hole.

6} The endoscope system according to claim 1, wherein the portion of the guide wire three-dimensionally bent is disposed while being decentered with respect to the guide through hole in a direction intersecting the longitudinal axis of the guide through hole.

7} The endoscope system according to claim 1, further comprising:
an endoscope adaptor that has the projecting portion and the guide through hole.

8} The endoscope system according to claim 1, wherein the projecting portion has an endoscope through hole through which the endoscope is passed and is detachably attached to the insertion portion.

9} The endoscope system according to claim 7, wherein the endoscope adaptor has a tubular sheath through which the endoscope is passed, and
the projecting portion is provided at a distal end portion of the sheath.

10} The endoscope system according to claim 9, wherein the sheath has a sheath bending portion that causes the sheath to bend, the sheath bending portion being disposed near a distal end of the sheath.

11} The endoscope system according to claim 1, wherein the projecting portion is integrally fixed to the insertion portion.
